(19) United States
(12) Patent Application Publication
(10) Pub. No.: US 2021/0213108 A1
(43) Pub. Date: Jul. 15, 2021

(54) METHODS AND COMPOSITIONS FOR RESECTION MARGIN LAVAGE

(71) Applicant: Stitch Bio, LLC, Waltham, MA (US)

(72) Inventors: Thomas C. Meyers, Dover, MA (US); Anthony P. Shuber, Northbridge, MA (US)

(21) Appl. No.: 17/147,709

(22) Filed: Jan. 13, 2021

Related U.S. Application Data
(60) Provisional application No. 62/960,393, filed on Jan. 13, 2020.

(51) Int. Cl. A61K 38/46; A61K 47/69

(52) U.S. Cl. CPC A61K 38/46; A61K 47/6929

(57) ABSTRACT

This disclosure provides methods and compositions for treating cancer. The invention relies on genome-editing tools to selectively target and kill cancer cells while minimizing deleterious effects to the subject. The genome-editing tools are designed to target and act on specific sequences identified in a genome of a tumor cell and absent from a genome of a healthy cell from the same patient. This specificity allows the genome-editing tool to target and kill cancer cells at the edge or border of a surgical site where a tumor was removed while leaving healthy cells unharmed.

### TECHNICAL FIELD

The disclosure relates to methods and compositions for treating cancer.

### BACKGROUND

For many cancer patients, tumor resection is a part of a treatment program. Tumor resection involves the surgical removal of a tumor and a margin of apparently normal tissue that surrounds the tumor to ensure that all cancer cells are removed. However, even after careful surgery, cancer cells may be left behind, threatening the possibility of disease relapse and cancer mortality.

In addition to surgery, many patients are now also treated with a combination of therapies involving toxic chemotherapeutic drugs and/or radiation therapy. One difficulty with this approach, however, is that radiotherapeutic and chemotherapeutic agents are toxic to normal tissues, and often create life-threatening side effects.

### SUMMARY

This disclosure provides methods and compositions for treating a surgical site to kill cancer cells left behind after tumor resection. Genome-editing tools are used to target and kill the cancer cells and are preferably delivered in a protein format, as an active nuclease, to avoid systemic uptake and circulation and thus minimize deleterious effects to the subject. The genome-editing tools are designed to target and cleave sequences specific to a tumor genome and absent from a genome of a healthy cell from the same patient. This specificity allows for the targeted destruction of cancer cells while leaving tissues cells unharmed.

Methods and compositions of the invention provide for the targeted delivery of genome-editing tools, such as nucleases, to specific sequences present in a cancer cell. For example, in some embodiments, this disclosure relies on clustered regularly interspaced short palindromic repeats (“CRISPR”) associated protein, or “Cas” endonucleases complexed with guide RNA. Through the use of a guide RNA, the Cas endonuclease complex is directed to desired locations in the genome. This specificity allows the Cas endonucleases to target and kill cancer cells while leaving healthy cells unharmed.

Methods of the invention include applying a composition in situ comprising a nuclease that cleaves DNA in target cancer cells that are present at the site of a surgical tumor resection. The nuclease is designed to act on sequences found specifically in the genome of a cancer cell and not also in corresponding portions of matched normal sequences from the same patient. In cancer cells, the nuclease targets and cleaves cancer-specific sequences while in normal cells, the nuclease is inert. In certain instances, once the nuclease has acted on the cancer-specific sequence, an apoptotic response is triggered and the target cancer cell will die.

Methods of the invention include inducing death of a target cancer cell with nucleases of the invention. In some aspects, cutting cancer-specific sequences with nucleases results in the destruction of cancer DNA and causes the target cell to die. In other aspects, nucleases of the invention are used to insert and integrate exogenous coding sequences, e.g., by homology-directed end repair, into the genome of the target cancer cell. The exogenous coding sequences may be provided as an expression cassette with regulatory sequences such as promoters or transcription factor binding sites that induce expression of those coding sequences. Inducing expression of the exogenous coding sequences in vivo can be used to cause the destruction of target cancer cells. For example, expression of exogenous sequences may modulate expression of cell cycle or apoptotic genes, for example, to cause cell death via apoptosis. In other instances, expression of exogenous sequences may produce cell-surface proteins on the surface of cancer cells that function as neoantigens. Expression of neoantigens may be used to mark the target cancer cells for death by, for example, the immune system or an antibody-drug-conjugate.

In some aspects, methods of the invention include treating a site of a surgical tumor resection with a composition that includes a nuclease in the format of an active ribonucleoprotein (RNP). Delivering the nuclease as an active protein complex is advantageous because the size of the RNP complex inhibits systemic uptake and circulation, thus reducing the statistical probability of an off-target effect. The composition may be provided as a lavage, or a similar therapeutic composition used as a surgical rinse. In some instances, the composition contains inert diluents, such as, for example, saline, water, or other solvents, solubilizing agents and emulsifiers. The composition may be introduced to the resection margin during or after surgery and may be used to wash away cell debris broken apart during surgery to prevent the possibility of cells from the resected tumor from seeding back into marginal tissue.

In other aspects, methods of the invention will include, prior to resecting the tumor, obtaining a biopsy from a subject containing tumor DNA and analyzing the tumor DNA (e.g., by NGS sequencing methods) and identifying a target in the tumor DNA that is absent in DNA of a healthy, non-tumor cell of the subject. For example, methods may include sequencing normal DNA taken from a healthy, non-tumor cell of the subject to thereby obtain normal sequences to compare with tumor sequences and identify tumor-specific sequences. Methods may include aligning the tumor sequences to matched normal sequences and identifying a target as a section of the tumor sequence that is absent from the matched normal sequences. Sequences appearing exclusively in the tumor genome may be identified as targets suitable for targeting with genome-editing tools.

As mutations accumulate in tumor DNA, the tumor genome becomes increasing unstable, causing harmful genomic rearrangements that include exchanges of DNA sequences between different chromosomal regions. Such chromosome rearrangements play a causal role in tumorigenesis by, for example, contributing to the inactivation of tumor-suppressor genes, dysregulated expression or amplification of oncogenes, and generation of novel gene fusions. In some embodiments, methods of this disclosure exploit the connection between fusion sequences and tumor genomes by targeting genome-editing nucleases to particular fusion sequences. Once the genome-editing nuclease encounters the fusion sequence, the nuclease will cleave the DNA causing the target cell to die.

A genome-editing nuclease may be designed to hybridize specifically to a region of a target cancer cell's genome that contains a fusion sequence, e.g., a gene fusion. The design of the guide RNA is preferably, but not necessarily, driven by sequencing nucleic acid corresponding to a tumor (e.g., cells from a biopsy) to determine where genomic instability (e.g., chromosomal rearrangement) has occurred. Because fusions are a phenotype of an unstable genome, targeting fusion sequences with guide RNA provides an optimal method for the targeted destruction of unhealthy cells while minimizing deleterious effects to the subject.

In preferred embodiments, a genome-editing tool is a Cas endonuclease complexed with a guide RNA, wherein the guide RNA includes the targeting sequence. In other embodiments, the genome-editing tool includes at least one transcription activator-like effector nuclease (TALEN) with a primary amino acid sequence that confers target specificity on the TALEN to a target in the genome of a tumor cell in a subject. In other embodiments, the genome-editing tool is a zinc-finger nuclease.

Methods of the invention include inducing death of a cancer cell using genome-editing systems. The method may include identifying a target sequence in tumor DNA of a subject and delivering one or more vectors comprising a genome-editing system to the subject. For example, a first vector may include DNA encoding a guide RNA that is capable of hybridizing with the target sequences. The vector may also include DNA encoding a Cas-related endonuclease, or alternatively, the Cas endonuclease may be encoded by a second vector delivered simultaneously with the first vector. The genome-editing system may include a Cas endonuclease that targets and cleaves one or more tumor-specific sequences resulting in cell death of the target cancer cell. In some instances, the genome-editing system may provide for the insertion and integration of an exogenous coding sequence, e.g., homology-directed repair, into the tumor genome. The exogenous coding sequence may be provided as an expression cassette in combination with the one or more vectors and may contain regulatory sequences, such as a promoter or transcription factor binding site, that induce expression of the exogenous coding sequence upon incorporation into the target genome. Targeted expression of exogenous coding sequences may then be used to kill target cancer cells.

In some aspects, methods of the invention include introducing to a resection margin or surgical margin nuclease protein complexes that harbor certain receptor ligands designed to drive the internalization of the nuclease by cells. For example, the nuclease protein complex may comprise a Cas protein complexed with guide RNA, wherein the Cas protein complex harbors surface exposed cysteines, for example C547, allowing for ligation to pyridyl disulfide-activated ligands.

In other aspects, methods of the invention include delivering a RNP including a nuclease complex to margins of a surgical resection by lipid particles. For example, lipid particles may include solid lipid nanoparticles or liposomes. For example, following a tumor resection, a composition may be introduced to the resection margin, wherein the composition includes dozens, or several hundred, or several thousand lipid nanoparticles packaging at least a corresponding number of the RNP. The lipid nanoparticles may be packaged in a vessel or container such as a blood collection tube or a microcentrifuge tube. For example, in some embodiments, the container comprises a microcentrifuge tube. The lipid nanoparticles may be provided as an aqueous suspension in one or more such containers.

In certain aspects, methods of the invention include introducing a composition to a resection margin that contains a mixture of nuclease complexes, such as, Cas endonuclease, wherein each complex is targeted to a different tumor-specific sequence, e.g., various fusion sequences. For example, a mixture of nuclease complexes may be packaged inside, or embedded within, carriers inside the composition wherein each complex includes a guide RNA directing the nuclease to a different fusion sequence. This is advantageous due to the fact that not all cancer associated fusion sequences identified within a cancer genome will feature a recognition site necessary for the nuclease complexes to recognize and bind to the tumor DNA. Providing a mixture of nuclease complexes increases the statistical likelihood that a particular nuclease complex will bind to a target sequence and induce cell death of the cancer cell.

In other aspects, methods of the invention provide an approach for treating a resection margin comprising the steps obtaining a biopsy from a patient, identifying a fusion sequence in DNA taken from the biopsy, and delivering to the patient a composition containing a RNP comprising a nuclease, such as, a Cas endonuclease complexed with guide RNA, wherein the guide RNA is capable of hybridizing with the fusion sequence identified in the DNA taken from the patient biopsy. In some embodiments, the composition may be provided as a lavage that includes a carrier for the RNP. The carrier may be a lipid nanoparticle comprising cationic lipids to facilitate the delivery of the RNP into target cells upon administering the composition to, for example, a resection margin.

In some aspects, methods of the invention include introducing a lavage to a resection margin wherein the lavage includes a genome-editing system, such as, a Cas endonuclease complexed with a guide RNA that includes a targeting sequence. The Cas endonuclease and guide RNA may be provided as a RNP. The RNP may be packaged in one or more nanoparticles for delivery, for example, the RNP may be packaged or embedded within lipid nanoparticles comprising cationic lipids in order to facilitate the delivery of the RNPs into target cells.

In certain aspects, this disclosure provides a use of a Cas-associated protein in making a medicament for a lavage. The use may further include providing the Cas-associated protein with guide RNA having a sequence that is substantially complementary to a fusion sequence of a target cancer cell. In other embodiments, the use may include providing the Cas-associated protein as an RNP to be introduced to a resection margin by a lavage.

In other aspects, this disclosure provides a composition for treating a tumor resection margin. The composition including a ribonucleoprotein (RNP) comprising a Cas endonuclease that cuts genomic DNA in a target cell to kill the target cell. The Cas endonuclease complexed with a guide RNA. The composition may further include a carrier to facilitate topical delivery of the RNP, wherein the carrier may include a gel or an ointment. In other embodiments, the composition is a lavage. The composition may be provided an aqueous suspension with the RNP suspended in an aqueous carrier. The composition may further comprise a lipid nanoparticle having the RNP packaged or embedded therein. The guide RNA, of the Cas complex, may include a recognition sequence substantially complementary to a target sequence comprising a gene fusion present in tumor DNA taken from a patient.

In other aspects, this disclosure provides a lavage for treating a resection margin. The lavage may contain a RNP including Cas endonuclease, e.g., Cas9, that is complexed with guide RNA. The lavage further includes a carrier for delivering the RNP to target tissue. For example, the RNP may be carried by nanoparticles or liposomes. In some embodiments, the lavage may include dozens, or several hundred, or several thousand lipid nanoparticles packaging at least a corresponding number of the RNP comprising Cas and guide RNA. The guide RNA may include a recognition sequence substantially complementary to a target sequence comprising a fusion sequence, for example a gene fusion, identified in nucleic acid of a resected tumor. In some embodiments the lavage may comprise RNP having a size and half-life properties that prevent the RNP from entering a blood stream and negatively impacting off-target tissues.

### BRIEF DESCRIPTION OF THE DRAWINGS

FIG. 1 diagrams a method for treating a resection margin.

FIG. 2 diagrams a method for targeting a tumor specific sequence.

FIG. 3 shows identifying a tumor-specific sequence.

FIG. 4 shows a gene editing system comprising Cas endonuclease.

FIG. 5 shows a composition of the disclosure.

### DETAILED DESCRIPTION

This disclosure provides methods and compositions for treating cancer by delivering genome-editing nucleases to specific sequences present in a cancer cell or pre-cancerous cell. For example, in some embodiments, this disclosure relies on clustered regularly interspaced short palindromic repeats (“CRISPR”) associated protein, or “Cas” endonucleases complexed with guide RNA. Through the use of a guide RNA, the Cas endonuclease complex is directed to desired locations of a cancer genome. This specificity allows the Cas endonucleases to target and kill cancer cells at the edge or border of a surgical site where a tumor was removed. Guide RNAs may be designed based on differences identified between a mutated sequences found in the resected tumor and a wild-type sequence obtained from a healthy cell of the same patient.

Mutations in genomic DNA can lead to genomic instability and eventually result in cancer. There are a variety of treatment options for cancer patients. In some instances, removing the cancerous cells by surgery may be the patient's best treatment option. This is referred to as tumor resection or surgical resection. For this type of surgery, a surgeon makes an incision through skin, muscle, or sometimes bone, and removes the cancerous cells along with some surrounding healthy tissue to ensure that all of the cancer is removed. However, no matter how expertly the surgery is performed, sometimes residual cancer cells are left behind. Moreover, there is a danger of spreading cancerous cells during a tumor resection (called seeding). Because cancer cells can metastasize and implant elsewhere in the body, the surgeon must minimize the dissemination of cells throughout the operating field or into the blood stream.

The resection margin is the margin of apparently non-tumorous tissue around a surgical site where a tumor that has been removed, referred to as the resected. The resection is an attempt to remove a tumor so that no portion of the malignant growth extends past the edges or margin of the removed tumor and surrounding tissue. These are retained after the surgery and examined microscopically by a pathologist to see if the margin is indeed free from tumor cells. If cancerous cells are found at the edges the operation is much less likely to achieve the desired results.

Sometimes, additional treatments are used following the operation to kill cancerous cells that might be left behind following surgery, such as, radiation, and chemotherapy. Often, these therapies act by targeting and killing cells of the body that divide rapidly. But these therapies also kill normal, rapidly dividing cells, such as hair follicles, cells of the digestive tract, and bone marrow. Thus, there is a problem with those therapies is that they are non-specific for targeting a cancerous cell and killing many normal cells. While killing the cancerous cells, collateral damage and death to the normal cells can result in other deleterious effects to the patient, for example, loss of hair, blood disorders such as leucopenia, digestive disorders, and physical pain.

This disclosure provides improved methods and compositions for treating cancer. In particular, methods of this disclosure provide a treatment for a resection margin following tumor removal. Methods include introducing a composition to the resection margin that comprises a genome-editing tool, for example, a nuclease, designed to kill residual cancer cells. Nucleases provided by this disclosure selectively target and kill cancer cells left behind following a tumor resection and leave normal, healthy cells unharmed.

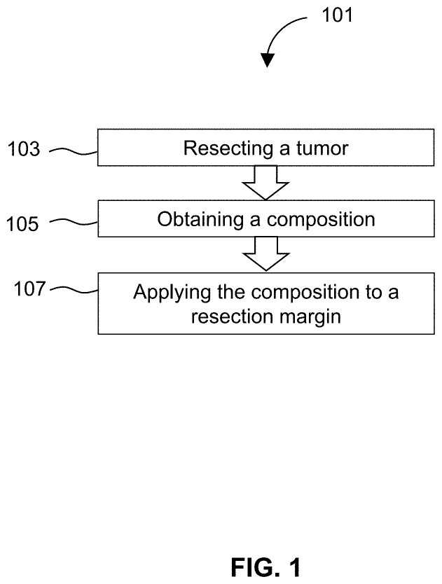

FIG. 1 diagrams a method for treating a resection margin. The method includes obtaining a composition 105 after resecting a tumor 103, then applying the composition 105 to the resection margin 107. The composition 105 includes a genome-editing tool, such as, a nuclease, that cleaves DNA in target cells present at the resection margin thereby causing death of the target cells. Preferably, the target cells are cancer cells that persist at the resection margin after tumor resection 103. To this end, the nuclease is designed to cleave DNA in cancer cells. For example, nucleases may target and cleave at genomic sequences found specifically in cancer cells and absent from a normal healthy cell, thereby inducing apoptosis or cell death in the cancer cell and leaving a normal, healthy cell unharmed. Preferably, the nuclease is provided as a RNP, and the composition 105 contains a carrier with the RNP packaged or embedded therein.

The composition 105 may be introduced or applied 107 to target tissue by a number of suitable methods which may depend at least partially on the chemical formulation of the composition 105. Preferably, the composition 105 is formulated for topical application, such as, for example, an oil, liquid, gel, or ointment and, upon application to the target margin, exhibits a beneficial local penetration and distribution. In some instances, the composition 105 is provided as a lavage, or similar surgical rinse, so that when applied 107, the lavage rapidly fills and occupies crevasses within the tissue of the cavity to deliver therapeutic compounds to target cells. The lavage may also be beneficial for washing away cell debris, for example, by using a syringe to repeatedly dispense and draw up the lavage within the resection margin. Washing the restricted margin may remove cell debris and prevent cells of the resected tumor from seeding back into the marginal tissue.

In some instances, the composition contains inert diluents, such as, for example, saline, water or other solvents, solubilizing agents and emulsifiers such as an alcohol. In some embodiments, the composition may further include any one of ethyl alcohol, isopropyl alcohol. The lavage may include ethyl carbonate, ethyl acetate, benzyl alcohol, benzyl benzoate, propylene glycol, 1,3-butylene glycol, dimethylformamide, an oil, glycerol, tetrahydrofurfuryl alcohol, polyethylene glycols and fatty acid esters of sorbitan, and mixtures thereof.

In other embodiments, methods of the invention may include providing a composition comprising a cream or ointment, and in which case, the composition may be topically administered by scooping up a portion of composition and rubbing the composition onto the target tissue. In other instances, the composition may comprise a gel that can be administered by a syringe by, for example, inserting the syringe into a body cavity at the site the tumor was removed and dispensing the composition directly onto the marginal tissue. In some instances, the composition may be administered as a spray by, for example, an aerosol canister.

Methods of the invention include introducing to a site of a surgical tumor resection a composition comprising a genome-editing tool, such as a nuclease, in a protein format, for example, as a RNP. The genome-editing nuclease may be, for example, a Cas endonuclease complexed with guide RNA. The Cas endonuclease may be, for example, Cas9 (e.g., spCas9), Cpf1 (aka Cas12a), C2c2, Cas13, Cas13a, Cas13b, e.g., PsmCas13b, LbaCas13a, LwaCas13a, AsCas12a, PfAgo, NgAgo, CasX, CasY, others, modified variants thereof, and similar proteins or macromolecular complexes. Delivering the nuclease as an active protein complex provides several benefits. For example, RNPs are often incorporated into cells that are difficult to transfect, such as stem cells. Also, delivery of a RNP does not require introducing foreign DNA into a subject, which limits the potential for off-target effects since the RNP is degraded over time. Moreover, because of their size, RNPs are inhibited from entering blood streams of a subject, thereby further reducing the statistical probability of an unwanted off-target effect.

The nuclease preferably includes one or more nuclear localization signals (NLSs) to promote migration of the nuclease to the nucleus of tumor cells. NLSs are short polypeptide sequences, e.g., about 10 to 25 amino acids long, and the sequences may be determined by searching literature, e.g., searching a medical library database for recent reports of nuclear localization signals.

In a preferred embodiment, the genome-editing nuclease comprises a Cas endonuclease. Cas is an RNA-guided endonuclease that is useful for in a genome-editing system. Included with the Cas endonuclease are guide RNA, which include two short single-stranded RNAs, the CRISPR RNA (crRNA), which is customizable and enables specificity for a target genetic material, and the trans-activating RNA (tracrRNA); although, those two RNAs are commonly provided as a single, fused RNA sometimes called a single guide RNA. As used herein, guide RNA refers to either format. Cas endonuclease and guide RNA form a RNP complex and bind to genomic DNA. In particular, the Cas complex stochastically scans the target genome to identify a protospacer adjacent motif (PAM) and then a genomic DNA sequence adjacent to PAM that matches the guide RNA sequence to cleave it. Thus, by virtue of a customizable sequence of the guide RNA, a Cas RNP may cleave target genetic material in a specific and controllable manner. Within the context of this disclosure, the specificity of the Cas9 proteins provides a system for inducing cell death of cancerous or pre-cancerous cells of a resection margin and leaving normal cells unharmed.

Nucleases used for methods and compositions of this disclosure may be purchased commercially. For example, Cas endonucleases may be purchased from a reagent distributor, such as, New England Biolabs. In other instances, nucleases according to this disclosure may be generated by in vitro transcription methods. In which case, plasmids encoding the nucleases may be purchased from, for example, Addgene, Inc.

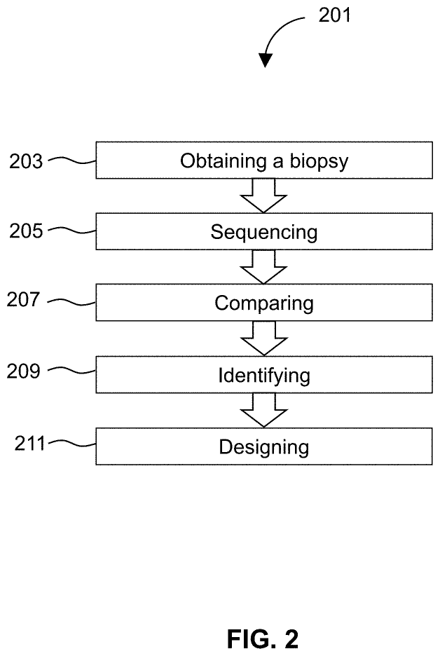

FIG. 2 diagrams a method for targeting a tumor specific sequence. Preferably, these steps will occur at least before the resecting step 103 of FIG. 1. The steps diagrammed in FIG. 2 include obtaining a biopsy 203 from a cancer patient. The biopsy 203 preferably includes genomic sequences of a similar composition as present in the tumor being surgically removed. In some embodiments, a second sample is also obtained at or near the time of biopsy 203 that includes healthy, non-tumor DNA. Healthy, non-tumor may comprise DNA taken from a cell identified as not being cancerous. The second sample may be obtained from a number of different sources, including blood or a cheek swab, of the cancer patient. Following the biopsy 203, the method includes sequencing 205 nucleic acids harvested from cells taken from the biopsy 203, comparing 207 those sequences to DNA sequences taken from the healthy, non-tumor cell. Comparing 207 may include aligning the tumor sequences to matched sequences taken from a healthy, normal cell and identifying 209 a target as a section of the tumor sequence that is absent from the matched normal sequences. Sequences appearing exclusively in the tumor genome may be identified 209 as the targets suitable for targeting with genome-editing tools. In preferred embodiments, target sequences comprise a fusion sequence, e.g., a gene fusion. Methods further include designing 211 guide RNA having a recognition sequence that is substantially complementary to nucleic acid taken from the biopsy 203. The recognition sequence is the specific sequence that recognizes the target DNA region of interest and directs the Cas endonuclease there for editing. In particular, the guide RNA will be designed 203 in order to bind to identified 209 sequences by complementary base pairing.

According to methods of this disclosure, tumor and matched-normal DNA may be sequenced (e.g., by a NGS sequencing instrument) 205 to generate tumor and matched-normal sequences 209. Methods for obtaining, identifying, and sequencing tumor and matched-normal DNA are well known in the art. For example, see methods described in U.S. Pub. 2013/0210645, U.S. Pub. 2004/0157243, U.S. Pat. No. 6,451,555, U.S. Pub. 2004/0157243, each of which is incorporated by reference.

Genomic information of a non-tumor sample taken from a subject may be compared 207 to genomic information of a tumor cell taken by biopsy, and tumor-specific genomic sequences may be identified 209 from the tumor sample. For example, the whole-genome sequence of tumor and matched-normal DNA may be compared 207. Tumor-specific genomic material may be identified 209 from the comparison 207, for example, by the appearance of sequence information present in the tumor specific sample and absent in the non-tumor sample, for example, presence of fusion sequences within the tumor specific sample. Comparing 207 may include comparing tumor sequences to matched-normal sequences (e.g., by alignment of assembled sequences from an NGS instrument run). The tumor-specific genomic material may include fusions sequence, for example, non-adjacent sequences present in the non-tumor sample, but detected as adjoining sequences in the tumor sample. The sequences that combine to produce a fusion sequence may originate from one or more chromosomes. Methods of the disclosure use the tumor-specific genomic material identified 209 to design 211 guide RNA that will selectively target a nuclease to a tumor specific sequence present in a tumor cell.

Sequencing may be performed by any method known in the art. For example, see, generally, Quail, et al., 2012, A tale of three next generation sequencing platforms: comparison of Ion Torrent, Pacific Biosciences and Illumina MiSeq sequencers, BMC Genomics 13:341. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing.

An example of a sequencing technology that can be used is Illumina sequencing. Illumina sequencing is based on the amplification of DNA on a solid surface using fold-back PCR and anchored primers. Genomic DNA is fragmented and attached to the surface of flow cell channels. Four fluorophore-labeled, reversibly terminating nucleotides are used to perform sequential sequencing. After nucleotide incorporation, a laser is used to excite the fluorophores, and an image is captured and the identity of the first base is recorded. Sequencing according to this technology is described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

Another example of a DNA sequencing technique that can be used is ion semiconductor sequencing using, for example, a system sold under the trademark ION TORRENT by Ion Torrent by Life Technologies (South San Francisco, Calif.). Ion semiconductor sequencing is described, for example, in Rothberg, et al., An integrated semiconductor device enabling non-optical genome sequencing, Nature 475:348-352 (2011); U.S. Pubs. 2009/0026082, 2009/0127589, 2010/0035252, 2010/0137143, 2010/0188073, 2010/0197507, 2010/0282617, 2010/0300559, 2010/0300895, 2010/0301398, and 2010/0304982, each incorporated by reference. DNA is fragmented and given amplification and sequencing adapter oligos. The fragments can be attached to a surface. Addition of one or more nucleotides releases a proton (H+), which signal is detected and recorded in a sequencing instrument.

Other examples of a sequencing technology that can be used include the single molecule, real-time (SMRT) technology of Pacific Biosciences (Menlo Park, Calif.) and nanopore sequencing as described in Soni and Meller, 2007 Clin Chem 53:1996-2001. Such sequencing methods are useful when obtaining large fragments of DNA from a reference or test sample, such as in the methods described in U.S. Pub. 2018/0355408, the contents of which are incorporated by reference herein.

In certain aspects, methods of this disclosure involve comparing sequence information obtained from a putative cancerous tissue from a patient with normal sequences from healthy tissue from the same patient in order to identify tumor-specific sequences for targeting nucleases. For example, in some aspects, methods may include using computer algorithms and software to align and match sequences obtained from tumor and normal cells to a reference genome, representative of a normal, healthy DNA. After the tumor and normal sequences are matched to the reference, methods may include identifying non-normal variations in the tumor sequence that does not appear in the matched-normal sequences. In some aspects, a threshold may be used to determine whether a portion of the tumor sequence should be classified as normal or determined as a non-normal variant, and thus identified as tumor-specific sequence. In some embodiments, any variation in the tumor sequence as compared to the matched-normal sequence may be identified as a tumor-specific sequence. While in other embodiments, variants specific to the tumor are identified based on their similarity or dissimilarity to the matched-normal sequence. For example, portions of the tumor sequence may be classified as tumor-specific sequence because it is varies from to a corresponding segment of the matched-normal sequence to a degree of 75%, 80%, 85%, 90%, 95%, 96%, 97%, 98%, 99%, 99.1%, 99.5%, 99.6%, 99.7%, 99.8%, 99.9%, etc. Methods for identifying tumor-specific sequences from patient tissue samples are well known in the art, and are described in U.S. Pub. 2016/0273049, U.S. Pub. 2012/0202207, U.S. Pub. 20150178445, U.S. Pub. 2019/0156922, incorporated here by reference.

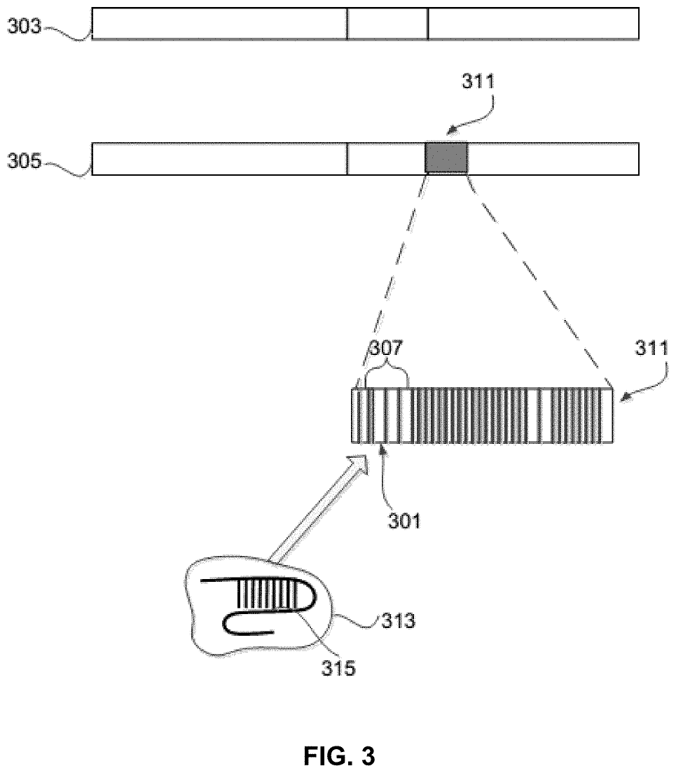

FIG. 3 illustrates a comparison of a matched-normal sequence 303 with a matched-tumor sequence 305 of DNA to identify a tumor-specific sequence 311. The analysis of a matched-tumor sequence 305 for identifying a tumor-specific target 311 can be used for targeting Cas endonucleases 313 to specific DNA sequences in cells of the resection margin. In the depicted embodiment, tumor sequence 305 is aligned to matched-normal sequences 303 to determine any differences. Where the tumor sequences 305 include tumor-specific genomic material 311 that are not also present in the matched-normal sequences 303, the tumor-specific genomic material 311 provides a target for cleavage by a gene editing system, in order to induce cell death of a cancer cell.

More particularly, in the depicted embodiment, a segment 307 of the tumor-specific genomic material 311 (e.g., DNA) is shown. The Cas endonuclease 313 is designed to recognize that segment and produce a double strand break in the DNA at the target 301. Because the matched-normal DNA does not include the tumor-specific sequence 311, a healthy, non-tumor genome does not include a corresponding segment 307 that cannot be recognized by the Cas endonuclease 313 and thus the Cas endonuclease 313 has no relevant effect on healthy, non-tumor cells.

In preferred embodiments, tumor specific material 311 comprises fusions sequences. A fusion sequence is a hybrid sequence formed from two previously separate sequences. It can occur as a result of: translocation, interstitial deletion, chromosomal inversion, chromosomal rearrangement, etc. Fusion sequences, such as gene fusions, comprises nucleic acid sequences that that occur separately on one or more chromosomes of normal healthy cell, and are present as a continuous, adjacent sequences in a tumor cell. Fusions are hallmarks of genome instability and therefore make suitable targets for tumor specific sequences 311.

A distinguishing feature of the segment 307 is that the segment 307 includes features that satisfy the targeting requirement of a gene-editing system such as Cas endonuclease 313. Thus, a distinguishing feature of the tumor-specific material 311 is that it is not also found in “matched normal” sequences from healthy, non-tumor cells. The segment 307 within the tumor material 311 includes matches for the targeting sequence of the gene-editing system. Where, for example, the gene editing system uses a Cas endonuclease 313, the segments 307 are those locations that include a suitable PAM adjacent to a suitable target sequence approximately 20 base target.

After identifying a target sequence 311, methods of this disclosure may further include designing a guide RNA having a recognition sequence substantially complementary to the target sequence 311, for example, a sequence of nucleic acid taken from a biopsy that is absent in sequences taken from a healthy, non-tumor cell. In some embodiments, the target sequence 311 may include a fusion sequence, for example, a gene fusion. Several software tools exist for designing an optimal guide with minimum off-target effects and maximum on-target efficiency. The following tools are the most popular guide RNA design tools available: Synthego Design Tool, Desktop Genetics, Benchling, and MIT CRISPR Designer. Once the guide sequence has been designed, the next step is to make it. This may be achieved by synthetically generating the guide RNA or by making the guide in vivo or in in vitro, starting from a DNA template.

In a preferred embodiment, the gene editing system uses Cas endonuclease and guide RNA. For example, the Cas endonuclease may be Cas9 from Streptococcus pyogenes (spCas9). The Cas endonuclease may be complexed with a guide RNA 315 as a RNP. One of skill in the art may design the guide RNA 315 to have a 20-base targeting sequence complementary to the segment 307 of the tumor-specific genomic material 311. Alternatively, the guide RNA 315 may have a 20-base targeting sequence complementary to a target within a few hundred or thousand bases of the segment 307. The target may be a sequence describable as 5′-20 bases-protospacer adjacent motif (PAM)-3′, where the PAM depends on Cas endonuclease.

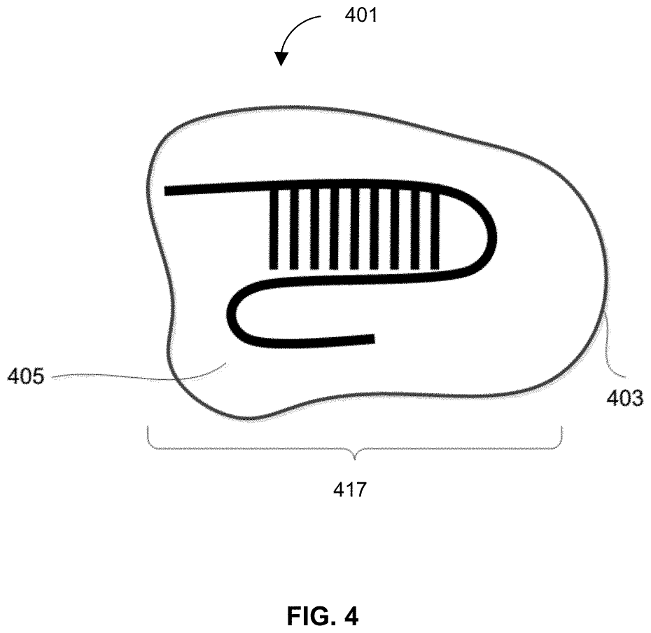

FIG. 4 shows an embodiment of a CRISPR-Cas system 401. The CRISPR-Cas system 401 relies on two main components: a guide RNA 405 and a CRISPER-associated (Cas) endonuclease 403. The guide RNA 405 is a specific RNA sequence that recognizes target DNA region of interest and directs the Cas endonuclease 403 there for editing. The guide RNA 405 is made up of at least two parts: crispr RNA (crRNA), a 17-20 nucleotide sequence complementary to the target DNA, and a tracr RNA, which serves as a binding scaffold for the Cas endonuclease 403. In particular, the crRNA of the guide RNA 405 includes a targeting sequence of approximately 17-20 bases complementary or nearly complementary to a target in tumor-specific genomic material of a subject. The Cas endonuclease 403 and gRNA 405 are complexed together into a ribonucleoprotein (RNP) 417. The CRISPR/Cas system 401 in a lavage or method of the disclosure may include at least one Cas endonuclease 403.

The RNPs comprising a CRISPR Cas system 401 may bind to their targets in tumor-specific DNA and introduce double stranded breaks. Introduction of double stranded breaks in DNA causes apoptosis of a target cancer cell. In some aspects, the CRISPR Cas system 401 may be designed to hybridize only to the region of the target genome that contains a fusion sequence identified in the tumor genome and absent from the genome of a normal, healthy cell of the subject. The design of the guide RNA 405 is preferably, but not necessarily, driven by sequencing nucleic acid in resected tumor (e.g., cells from a biopsy) to determine where genomic instability (e.g., chromosomal rearrangement) has occurred. Because fusions are a phenotype of an unstable genome, targeting fusion sequences with guide RNA 405 provides methods more likely to target and kill unhealthy cells while minimizing deleterious effects to the subject.

Methods of the invention include inducing death of a target cancer cell with nucleases of the invention. In certain instances, simply cutting cancer-specific sequences with nucleases results in the destruction of cancer DNA and causes the target cell to die. In other instances, nucleases of the invention are used to insert and integrate exogenous coding sequences, e.g., by homology-directed end repair, into the genome of the target cancer cell. See How, 2019, Inserting DNA with CRISPR, Science 365(6448):25 and Strecker, 2019, RNA-guided DNA insertion with CRISPR-associated transposases, Science 365(6448):48, both incorporated herein by reference. The exogenous coding sequences may be provided as an expression cassette with regulatory sequences such as promoters or transcription factor binding sites that induce expression of those coding sequences. Induced expression of the exogenous coding sequences in vivo can be used to cause the destruction of target cancer cells. For example, expression of exogenous sequences may modulate expression cell cycle proteins such as cyclins and cyclin-dependent kinases (CDKs), to disrupt proliferation of target cancer cells or induce cell death. See Otto, 2017, Cell cycle proteins as promising targets in cancer therapy, Nat Rev Cancer 17(2): 93-115, incorporated herein by reference. Alternatively, expression of exogenous sequences may be used to modulate expression of certain apoptotic genes, for example, exogenous sequences may be used to upregulate caspase-9 expression to cause cell death via apoptosis. In other instances, expression of exogenous sequences may produce cell-surface proteins on cancer cells that function as neoantigens. Expression of neoantigens may lead to the expression of antigens that can be used to mark the target cancer cells for death by the subject's immune system, for example, as discussed in co-owned, and co-pending, U.S. Application 62/927,265, which is incorporated by reference. The insertion site of the exogenous sequence may be near the promoter region of a target gene. In some embodiments, the target site may be within an open reading frame (ORF) in the tumor-specific genomic material, and genome editing nuclease can integrate the exogenous coding sequence, in-frame, within the ORF. Insertion of the coding sequence into the ORF causes expression of the coding sequence. Gene editing systems can be designed and synthesized or ordered by making reference to the predetermined site in the tumor-specific genomic material.

In certain embodiments, the gene editing system includes a RNP that comprises a Cas endonuclease and a guide RNA, i.e., in which the guide RNA includes the targeting sequence. In other embodiments, the gene editing system includes at least one transcription activator-like effector nuclease (TALEN) with a primary amino acid sequence that confers target specificity on the TALEN to a target in the genome of the tumor cell in the subject.

In certain embodiments, methods include introducing a composition to a resection margin.

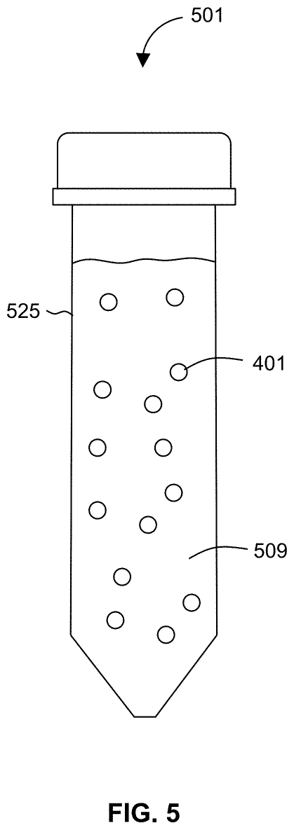

FIG. 5 shows a composition 501 for treating a tumor resection margin. The composition 501 includes a ribonucleoprotein (RNP) 401 comprising a Cas endonuclease that cuts genomic DNA in a target cell to kill the target cell. The Cas endonuclease is preferably complexed with a guide RNA. The composition 501 preferably also includes a carrier 509 for topical delivery of the RNP 401, such as a gel or an ointment. Optionally, the carrier 509 provides an aqueous suspension with the RNP 401 suspended in an aqueous carrier. In some embodiments, the composition 501 includes one or more a lipid nanoparticles having the RNP packaged or embedded therein. The composition 501 is preferably packed in a suitable vessel or tube 525, such a collection tube, test tube, or microcentrifuge tube. In preferred embodiments, the composition 501 contains a carrier with a gene editing system, such as, a Cas endonuclease and a guide RNA with a targeting sequence. The Cas endonuclease and guide RNA may be provided as an RNP embedded within the carrier. The carrier may be a nanoparticle, for example, a lipid nanoparticle comprising cationic lipids which may facilitate the delivery of the RNPs into target cells.

The nuclease preferably includes one or more nuclear localization signals (NLSs) to promote migration of the nuclease to the nucleus of target cancer cells. Even when the nuclease is provided in a nucleic acid, e.g., in mRNA or DNA sense, it still may include the NLSs, in frame with the ORF for the nuclease. NLSs are short polypeptide sequences, e.g., about 10 to 25 amino acids long, and the sequences may be determined by searching literature, e.g., searching a medical library database for recent reports of nuclear localization signals.

In other aspects, methods of the invention include introducing to a resection margin or surgical margin nuclease protein complexes that harbor certain receptor ligands designed to drive the internalization of the nuclease by specific cell types. For example, the nuclease protein complex may comprise a Cas protein complexed with guide RNA, wherein the Cas protein complex harbors a surface exposed cysteine, for example C547, allowing for ligation to pyridyl disulfide-activated ligands. In other embodiments, this disclosure provides RNPs comprising Cas nucleases with certain ligand-binding domains for nuclear receptors to facilitate the transport of Cas into the nucleus of a target cell.

In some aspects, methods of the invention include delivering a carrier comprising RNP having Cas endonuclease complexed with a guide RNA to margins of a surgical resection by lipid particles. For example, lipid particles may include solid lipid nanoparticles or liposomes. For example, following a tumor resection, a composition may be introduced to the resection margin, wherein the composition includes dozens, or several hundred, or several thousand lipid nanoparticles packaging at least a corresponding number of the RNP. The lipid nanoparticles may be packaged in a vessel or container such as a blood collection tube or a microcentrifuge tube. For example, in some embodiments, the container may comprise a microcentrifuge tube. The lipid nanoparticles may be provided in an aqueous suspension in a suitable container.

Methods of the invention may also include inhibiting tumor growth or metastasis of cancer in a subject by administering to the subject a therapeutically effective amount of a composition disclosed herein. A therapeutically effective amount of the composition disclosed herein is an amount sufficient to inhibit growth, replication or metastasis of cancer cells, or to inhibit a sign or a symptom of the cancer. The therapeutically effective amount may depend on disease severity, the type of disease, or the subject's general health. In general, methods include administering a therapeutic effective amount of the composition to a resection margin following surgical resection.

In some embodiments methods include introducing a composition to a resection margin wherein the composition contains a mixture of nuclease complexes wherein each complex is targeted to a different fusion sequence. For example, a mixture of nuclease complexes may be packaged inside the composition wherein each complex includes a guide RNA directing the nuclease complex to a different fusion sequence. This is advantageous due to the fact that not all fusion sequences identified within a cancer genome will have the recognition site necessary for the nuclease complexes to recognize and bind to the tumor DNA. By creating a mixture of nuclease complexes, it will increase the statistical likelihood of a particular nuclease complex binding to a target sequence and inducing cell death.

In other aspects, the disclosure provides a lavage for treating a resection margin. The lavage contains a carrier comprising a RNP with Cas endonuclease, e.g., Cas9, that is complexed with guide RNA. For example, the RNP may be carried by nanoparticles or liposomes. In some embodiments, the lavage may include dozens, or several hundred, or several thousand lipid nanoparticles packaging at least a corresponding number of the RNP comprising Cas and guide RNA. The guide RNA may include a recognition sequence substantially complementary to a target sequence comprising a fusion sequence, for example a gene fusion, identified in nucleic acid of a resected tumor. In some embodiments the lavage may comprise RNP having a size and half-life properties that prevent the RNP from entering a blood stream and negatively impacting off-target tissues.

Embodiments of the invention use any suitable gene editing system such as, for example, CRISPR systems, transcription activator like effector nucleases (TALENs), zinc finger nucleases, or meganucleases.

Methods of this disclosure may include introducing a composition to the resection margin, wherein the composition comprises a genome-editing nuclease. The nuclease may be provided as a protein, a RNP, mRNA, or by delivering DNA vectors such as plasmids or AAV vectors that encode the nuclease. The nucleic acid encoding the nuclease may be introduced into the cell by a variety of means, for example, a clonal micelle, liposome, extracellular vesicle, nanoparticle, copolymer block, adeno-associated virus, virus-like particle, and adenovirus. Where, for example, the nucleases are Cas-type nucleases, such as Cas9 and variants thereof, DNA vectors may each encode a guide RNA complementary to the nucleic acid target, wherein the nuclease forms a complex with the guide RNA to specifically cut the target site, such as an identified fusion sequence.

In other aspects, this disclosure provides a composition for treating a resection margin following the surgical removal of a tumor. The composition may contain a carrier with a RNP, such as, a Cas endonuclease, e.g., Cas9, that is complexed with guide RNA. The carrier may be a nanoparticle or a liposome. In some embodiments, the composition may include dozens, or several hundred, or several thousand carriers such as lipid nanoparticles that package at least a corresponding number of the RNP comprising Cas and guide RNA. The guide RNA may include a recognition sequence substantially complementary to a target sequence, for example, a target sequence comprising a fusion sequence, such as, a gene fusion, identified in nucleic acid of a resected tumor. In some embodiments the composition may comprise RNP having a size and half-life properties that prevent the RNP from entering a blood stream and negatively impacting off-target tissues.

In some aspects, methods of the invention use lipid nanoparticles (LNPs) such as solid lipid nanoparticles comprising a nuclease. LNPs may be about 100-200 nm in size and may optionally include a surface coating of a neutral polymer such as PEG to minimize protein binding and unwanted uptake. The nanoparticles are optionally carried by a carrier, such as water, an aqueous solution, suspension, or a gel. For example, LNPs may be included in a formulation that may include chemical enhancers, such as fatty acids, surfactants, esters, alcohols, polyalcohols, pyrrolidones, amines, amides, sulfoxides, terpenes, alkanes and phospholipids. LNPs may be suspended in a buffer. Lipid nanoparticles may be delivered via a gel, such as a polyoxyethylene-polyoxypropylene block copolymer gel (optionally with SLS). Poloxamers are nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist having different properties. For the generic term “poloxamer”, these copolymers are commonly named with the letter “P” (for poloxamer) followed by three digits: the first two digits×100 give the approximate molecular mass of the polyoxypropylene core, and the last digit×10 gives the percentage polyoxyethylene content (e.g. P407=poloxamer with a polyoxypropylene molecular mass of 4,000 g/mol and a 70% polyoxyethylene content). LNPs may be freeze-dried (e.g., using dextrose (5% w/v) as a lyoprotectant), held in an aqueous suspension or in an emulsification, e.g., with lecithin, or encapsulated in LNPs using a self-assembly process. LNPs are prepared using ionizable lipid L319, distearoylphosphatidylcholine (DSPC), cholesterol and PEG-DMG at a molar ratio of 55:10:32.5:2.5 (L319:DSPC:cholesterol:PEG-DMG). The payload may be introduced at a total lipid to payload weight ratio of ˜10:1. A spontaneous vesicle formation process is used to prepare the LNPs. Payload is diluted to ˜1 mg/ml in 10 mmol/l citrate buffer, pH 4. The lipids are solubilized and mixed in the appropriate ratios in ethanol. Payload-LNP formulations may be stored at −80° C. See Maier, 2013, Biodegradable lipids enabling rapidly eliminating lipid nanoparticles for systemic delivery of RNAi therapeutics, Mol Ther 21(8):1570-1578, incorporated by reference. See, WO 2016/089433 A1, incorporated by reference herein. Compositions of the disclosure may include a plurality of lipid nanoparticles having the gene editing system, and in some instances, exogenous coding sequences, embedded therein. In one embodiment, a plurality of lipid nanoparticles comprises at least a solid lipid nanoparticle comprising a RNP comprising Cas9 complexed with a guide RNA targeting a tumor-specific sequence. In another embodiment, a plurality of lipid nanoparticles comprises at least a solid lipid nanoparticle comprising a RNP with Cas9 complexed with a guide RNA that targets the CRISPR/Cas system to a locus within a predetermined site in tumor-specific genomic material of a subject, and an expression cassette comprising an exogenous coding sequence with the one or more vectors that may contain regulatory sequences, such as a promoter or transcription factor binding site, that induce expression of the exogenous coding sequence upon incorporation into the target genome.

Compositions of this disclosure are preferably formulated for topical delivery to a resection margin. Compositions may be provided as aqueous suspensions, oil suspensions, or emulsions. The aqueous suspensions may contain one or more compounds in admixture with excipients suitable for the manufacture of aqueous suspensions. Oily suspensions may be formulated by suspending the compound in suitable oil such as mineral oil, arachis oil, olive oil, or liquid paraffin. The oily suspensions may contain a thickening agent, for example beeswax, hard paraffin or acetyl alcohol.

The compositions may also be in the form of oil-in-water emulsions. The oily phase may be a lipid, a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

Compositions may include pharmaceutically acceptable carriers, such as sugars, for example, lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol, sorbitol, mannitol and polyethylene glycol; esters, such ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

1. A method of treating a tumor resection margin, the method comprising:
applying to the resection margin a composition comprising a nuclease that cleaves DNA in target cells present at the resection margin, thereby causing death of the target cells.

2. The method of claim 1, wherein the target cells are tumor cells that persists at the resection margin after tumor resection.

3. The method of claim 2, wherein the nuclease specifically cleaves tumor DNA in the tumor cells.

4. The method of claim 1, wherein the nuclease is a RNP comprising a Cas endonuclease complexed with a guide RNA that targets the RNP specifically to tumor DNA.

5. The method of claim 4, further comprising, prior to the applying step:
performing a biopsy to obtain the tumor DNA;
sequencing the tumor DNA; and
designing the guide RNA to have a recognition sequence that is substantially complementary to a target sequence in the tumor DNA.

6. The method of claim 5, wherein the target sequence includes at least a portion of a gene fusion specific to the tumor.

7. The method of claim 4, wherein the composition includes a carrier for delivery of the RNP.

8. The method of claim 7, wherein the carrier is a nanoparticle.

9. The method of claim 8, wherein the nanoparticle is a lipid nanoparticle comprising cationic lipids.

10. The method of claim 4, wherein the Cas endonuclease is Cas9.

11. The method of claim 4, wherein the RNP comprises size and half-life properties that inhibit the RNP from entering a blood stream and damaging off-target tissue.

12. The method of claim 5, wherein the recognition sequence of the guide RNA has a high specificity towards the gene fusion, thereby inhibiting the RNP from damaging off-target tissues.

13. The method of claim 1, wherein the composition is introduced to the tumor resection margin during surgery.

14. The method of claim 1, wherein the composition is provided as a lavage.

15. A composition for treating a tumor resection margin, the composition comprising:
a ribonucleoprotein (RNP) comprising a Cas endonuclease that cuts genomic DNA in a target cell to kill the target cell, the Cas endonuclease complexed with a guide RNA; and
a carrier for topical delivery of the RNP.

16. The composition of claim 15, wherein the carrier comprises a gel or an ointment.

17. The composition of claim 15, wherein the composition is an aqueous suspension with the RNP suspended in an aqueous carrier.

18. The composition of claim 15, wherein the carrier comprises a lipid nanoparticle having the RNP packaged or embedded therein.

19. The composition of claim 18, wherein the guide RNA includes a recognition sequence substantially complementary to a target sequence comprising a gene fusion present in tumor DNA taken from a patient.

20. The composition of claim 19, wherein the RNP comprises a short half-life that prevents the RNP from entering a blood stream and negatively impacting off-target tissues.
